(19) United States
(12) Patent Application Publication
(10) Pub. No.: US 2025/0010105 A1
(43) Pub. Date: Jan. 9, 2025

(54) SECONDARY REFLECTOR FOR IMPROVEMENT OF SHOCKWAVE THERAPIES

(71) Applicant: WaveVision GmbH, Vienna (AT)

(72) Inventor: Cyrill B. SLEZAK, Orem, UT (US)

(21) Appl. No.: 18/766,430

(22) Filed: Jul. 8, 2024

Related U.S. Application Data
(60) Provisional application No. 63/525,453, filed on Jul. 7, 2023.

(51) Int. Cl. A61N 7/00

(52) U.S. Cl. CPC A61N 7/00; A61N 2007/0078

(57) ABSTRACT

An acoustic shockwave receiver device for use in shockwave therapies includes an impedance-controlled layer, at least partly conformable against a user body part, and a terminating reflector directly coupled to the impedance-controlled layer. The terminating reflector has a predetermined fixed geometry selected to receive and redirect to a treatment zone of the user body part at least a majority of an acoustic shockwave received through the user body part and the impedance-controlled layer.

### CROSS REFERENCE TO RELATED APPLICATIONS

The present application claims benefit of priority to co-pending U.S. Provisional Patent Application No. 63/525,453, entitled SECONDARY REFLECTOR FOR IMPROVEMENT OF SHOCKWAVE THERAPIES, filed Jul. 7, 2023, which, to the extent not inconsistent with the disclosure herein, is incorporated by reference in its entirety.

### BACKGROUND

Shockwaves have been used in the medical field since the 1980's for the treatment of a wide range of indications. For example, in extracorporeal shockwave therapy (ESWT) device-generated shockwaves are directed at a target tissue within the device's so-called therapy zone. A shockwave applicator is acoustically coupled to the body part to be treated via an acoustical coupling gel (e.g., ultrasound gel). An acoustic shockwave is generated in the device which propagates through the coupling-gel layer into the body part where it continues to move toward the target tissue. This results in a biological response in the body.

In particular, millions of men globally experience erectile dysfunction, with most treatments offering only temporary relief. ESWT has been shown to promote neovascularization and to reduce dysfunction caused by poor cavernosal arterial blood flow. However, conventional ESWT includes risks of bleeding or bruising of the treated area, skin infection at a treatment location, and pain.

The mechanism for relief resulting ESWT is not entirely understood. This is believed to be, at least in part, due to lack of a consistent application and treatment standard. Conventional treatments reported in the literature differ dramatically in duration, number of repeat treatments, distance to target, intensity of shockwave, and condition of the treatment tissue (e.g., erect vs. flacid). Moreover, although shockwave applicators are designed to deliver an intended pressure pulse to the target tissue, the wave propagation beyond the target tissue is largely ignored in conventional therapy. After passing through the therapy zone, waves continue to propagate through the human body, beyond the target tissue, based on the body's anatomy. Of particular concern are areas where shockwaves passing through soft-tissue encounter a gaseous (e.g., air) boundary where they reflect (e.g., at the skin, lungs, trachea). The direction of the reflection corresponds to and/or is governed by the shape of the interface between tissues and gases. Treatment zones near these tissue-gas boundaries can result in medical complications such as induced pulmonary capillary hemorrhaging.

Further, the shape of these anatomical boundaries can result in undesirable and/or unpredictable reflections that affect other tissues. For example, if the shockwave encounters a boundary beyond the target tissue (e.g., soft-tissue/skin-to-air) of a concave (or concave-like) shape, the thus-reflected wave may be unintentionally re-focused, potentially resulting in even stronger shockwave effects as a strengthening of the shockwave occurs within the body. These reflected waves create additional, uncontrolled physical effects inside the treatment zone which may, depending on the remaining magnitude in the reflected waves, result in adverse biological responses.

### SUMMARY OF THE DISCLOSURE

This Summary introduces a selection of concepts in a simplified form in order to provide a basic understanding of some aspects of the present disclosure. This Summary is not an extensive overview of the disclosure, and is not intended to identify key or critical elements of the disclosure or to delineate the scope of the disclosure. This Summary merely presents some of the concepts of the disclosure as a prelude to the Detailed Description provided below.

According to an embodiment, a shockwave therapy treatment system includes a shockwave transducer, an impedance-controlled layer, and a shockwave receiver. The shockwave transducer is configured to produce an acoustic shockwave and direct the shockwave to a treatment area of a patient body part. The impedance-controlled layer is configured to be conformable against the patient body part at a location across the patient body part from the shockwave transducer through the treatment area. The shockwave receiver has a shape positioned against the impedance-controlled layer and is configured to receive at least a portion of the acoustic shockwave via the patient body part and the impedance-controlled layer and to affect one or more characteristics of the acoustic shockwave.

According to an embodiment, an acoustic shockwave receiver device for use in shockwave therapies includes an impedance-controlled layer and a terminating reflector. The impedance-controlled layer is at least partly conformable against a user body part. The terminating reflector is directly coupled to the impedance-controlled layer and has a predetermined fixed geometry selected to receive and redirect to a treatment zone of the user body part at least a majority of an acoustic shockwave received through the user body part and the impedance-controlled layer.

According to an embodiment, a method for treating a body part using acoustic shockwaves incudes an operation of identifying at least one treatment zone of a subject organism. The method further includes an operation of aligning an acoustic shockwave generator to a position proximate the treatment zone to direct and focus an acoustic shockwave to the treatment zone. The method further includes an operation of placing a non-gaseous impedance-controlled layer in direct acoustical contact with the organism at a location opposite the acoustic shockwave generator across the treatment zone. The method further includes an operation of terminating the impedance-controlled layer with a shockwave reflector having a predetermined fixed geometry, and configured to reflect the acoustic shockwave and focus the shockwave on a secondary treatment zone.

Further scope of applicability of the present invention will become apparent from the Detailed Description given below. However, it should be understood that the Detailed Description and specific examples, while indicating preferred embodiments of the present invention, are given by way of illustration only, since various changes and modifications within the spirit and scope of the present invention will become apparent to those skilled in the art from this Detailed Description.

### BRIEF DESCRIPTION OF DRAWINGS

These and other objects, features and characteristics of the present disclosure will become more apparent to those skilled in the art from a study of the following Detailed Description in conjunction with the appended claims and drawings, all of which form a part of this specification. In the drawings:

FIG. 1 illustrates a shockwave therapy treatment system, according to an embodiment.

FIG. 2 is a cross-sectional view of an impedance-controlled layer, according to an embodiment.

FIGS. 3A-3C show views of shockwave reflectors, according to an embodiment.

FIG. 4 is a perspective bottom view of a shockwave reflector, according to an embodiment.

FIG. 5 is a cross-section diagram illustrating the shockwave trajectory intended in a shockwave therapy session using the disclosed system, according to an embodiment.

FIG. 6 illustrates a method flow chart for using a secondary reflector in a shockwave therapy treatment system, according to an embodiment.

FIGS. 7-9 illustrate a testing setup and corresponding results in which a shockwave transducer was applied to a simulated body part, according to an embodiment. FIG. 7 includes photos of the test setup, according to an embodiment.

FIG. 8 includes acoustic peak-pressure simulation results showing shockwave propagation in different setups from FIG. 7, according to an embodiment. FIG. 9 includes respective graphs showing shockwave pressure vs. time as measured in the different setups, according to an embodiment.

The headings provided herein are for convenience only and do not necessarily affect the scope or meaning of the claimed invention.

In the drawings, the same reference numerals and any acronyms identify elements or acts with the same or similar structure or functionality for ease of understanding and convenience. The drawings will be described in detail in the course of the following Detailed Description.

### DETAILED DESCRIPTION

Various examples of the present invention will now be described. The following description provides specific details for a thorough understanding and enabling description of these examples. One skilled in the relevant art will understand, however, that the present invention may be practiced without many of these details. Likewise, one skilled in the relevant art will also understand that the present invention can include many other obvious features not described in detail herein. Additionally, some well-known structures or functions may not be shown or described in detail below, so as to avoid unnecessarily obscuring the relevant description.

Descriptions of well-known starting materials, processing techniques, components and equipment may be omitted so as not to unnecessarily obscure the present invention in detail. It should be understood, however, that the detailed description and the specific examples, while indicating (e.g., preferred) embodiments of the present invention, are given by way of illustration only and not by way of limitation. Various substitutions, modifications, additions and/or rearrangements within the spirit and/or scope of the underlying inventive concept will become apparent to those skilled in the art from this disclosure.

As used herein, the terms “comprises,” “comprising,” “includes,” “including,” “has,” “having” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations include, but is not limited to: “for example,” “for instance,” “e.g.,” “in one embodiment.”

It should be noted that while examples and embodiments disclosed herein may be directed to certain types of users, environments, etc. (e.g., erectile dysfunction), these limitations are only for purposes of ease of understanding and should not be deemed to limit the disclosure in scope. For example, the disclosed system is not limited to treatment of erectile dysfunction but instead may be adapted and applied to any market/industry; for example, the disclosed system may be applicable to treatments directed to healing of wounds, and other applications.

This disclosure discusses novel concepts for extracorporeal shockwave treatment (ESWT) which has been studied and/or employed for at least pain relief, tissue regeneration (including, protein biosynthesis and proliferation, vascularization, neuroprotection, and chondroprotection), bone healing, and calcified tendinopathy. (See Simplicio et al., “Extracorporeal Shock Wave Therapy Mechanisms in Musculoskeletal Regenerative Medicine”, Journal of Clinical Orthopaedics and Trauma, 11, 2020 S309-S318.) A pressure wave is a disturbance or series of disturbances in the form of a variation of the local pressure in a medium which has the ability to propagate through solids, liquids and gases. A shockwave is a non-linear type of pressure wave with a short rise time. Positive (compressive) and negative (tensile) phases of shockwaves exert certain effects on interfaces between various tissues and their different densities. Shockwaves may hit an interface between two different media upon which they may be partially reflected, partially pass through, and gradually become absorbed. The fraction of the reflection is determined by the difference in the adjoining media's impedances; gases in and around a body (e.g., air) tend to reflect shockwaves at such interfaces at close to 100%, while body soft-tissues (e.g., around and beyond a target treatment area) tend to mostly transmit the waves, eventually absorbing the shockwaves. During the negative or “tensile” phase (i.e., negative pressure), the shockwave can potentially generate cavitations (bubbles) in the tissue. The cavitation bubbles then implode with high speed, producing a second wave of shockwaves or micro-jets of fluid. (See id., at 2.3.) While it is theorized that some level of cavitation can be therapeutic in some applications when specifically directed, cavitation can cause undesirable tissue disruption including undesirable tissue damage. This disclosure is directed generally to focused ESWT rather than radial ESWT. However, the inventors recognize that the disclosed methods and devices for handling shockwaves beyond a target area are applicable to both types of treatment.

Whereas under conventional application of extracorporeal shockwave therapy (ESWT), sound propagation beyond the therapy zone is uncontrolled, the disclosed procedure and devices permit control of the reflected sound fields.

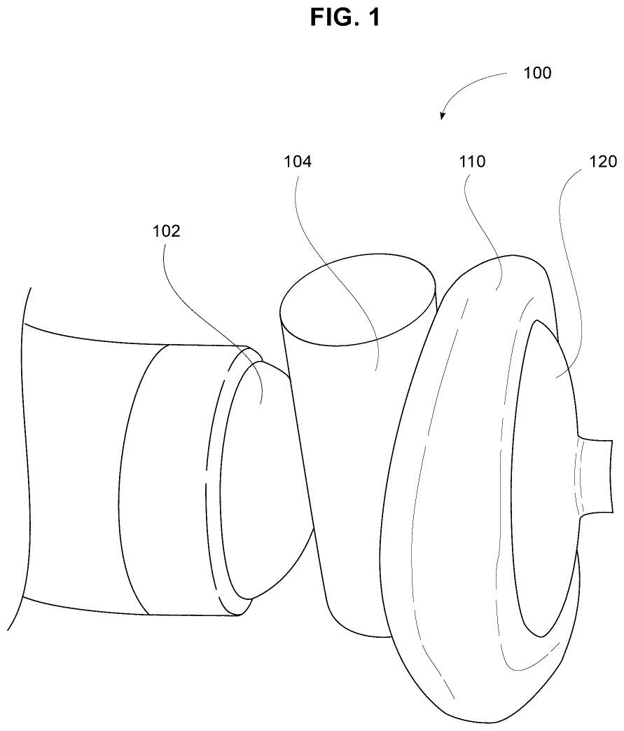

FIG. 1 illustrates a shockwave therapy treatment system 100 including a shockwave transducer 102, an impedance-controlled layer 110 and a shockwave receiver 120, according to an embodiment. The shockwave transducer 102 produces an acoustic shockwave and includes features to direct and focus the shockwave toward a treatment area of a patient's body part 104 (shown here as a cylinder). Those having skill in the art will acknowledge that a treated body part 104 may include any of various body appendages, including at least a penis, achilles tendon, an arm, a leg, fingers, toes, hands, feet, etc. In some applications, the system may be employed in a manner that either the transducer 102 or the impedance-controlled layer 110 and shockwave receiver 120 are placed in a cavity inside the patient body, e.g., in a trachea, colon, or the like in order to best target treatment of nearby body tissue(s).

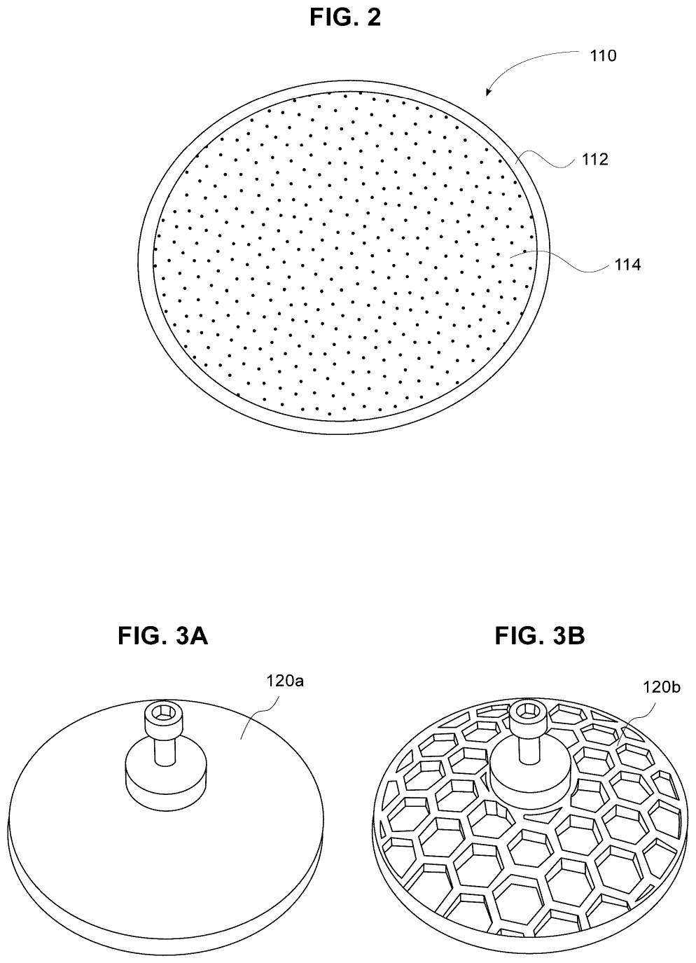

FIG. 2 is a cross-sectional view of an impedance-controlled layer 110, according to an embodiment. The impedance-controlled layer 110 may include a pouch 112 (e.g., an elastomeric shell) deformable against the patient's body part. According to an embodiment, the pouch/shell 112 may be formed from a material that is acoustically transparent or that at least minimally impedes acoustic waves. The pouch 112 is filled with an acoustically transparent material 114, which may be a liquid or gel. For example, the acoustically transparent material 114 may include a cohesive liquid selected for its resistance to cavitation. As discussed briefly above, cavitation can impact body tissues, but also result in significantly increased energy absorption/loss of the wave inside the impedance-controlled layer 110. Examples of fluids that resist cavitation for use in the impedance-controlled layer include, but are not limited to, one or more of: acoustic gels, high-viscosity mineral oil, water-glycol mixtures, silicone oils, certain polyalphaolefins (PAO) and/or hydraulic fluids with anti-cavitation additives. The inventor recognizes, however, that other fluids may be employed in appropriate circumstances. For example, water is employed in some types of ESWT due to its similar acoustic impedance to body tissues. Accordingly, saline could be employed in the impedance-controlled layer in appropriate circumstances. Those having skill in the art will recognize that non-liquid materials having an impedance similar to body tissues may be used within the scope of the invention. For example, certain perfluorocarbons (PFCs) and other gas mixtures engineered to have similar acoustic properties to body tissues in certain conditions could be used in the impedance-controlled layer within the scope of the invention.

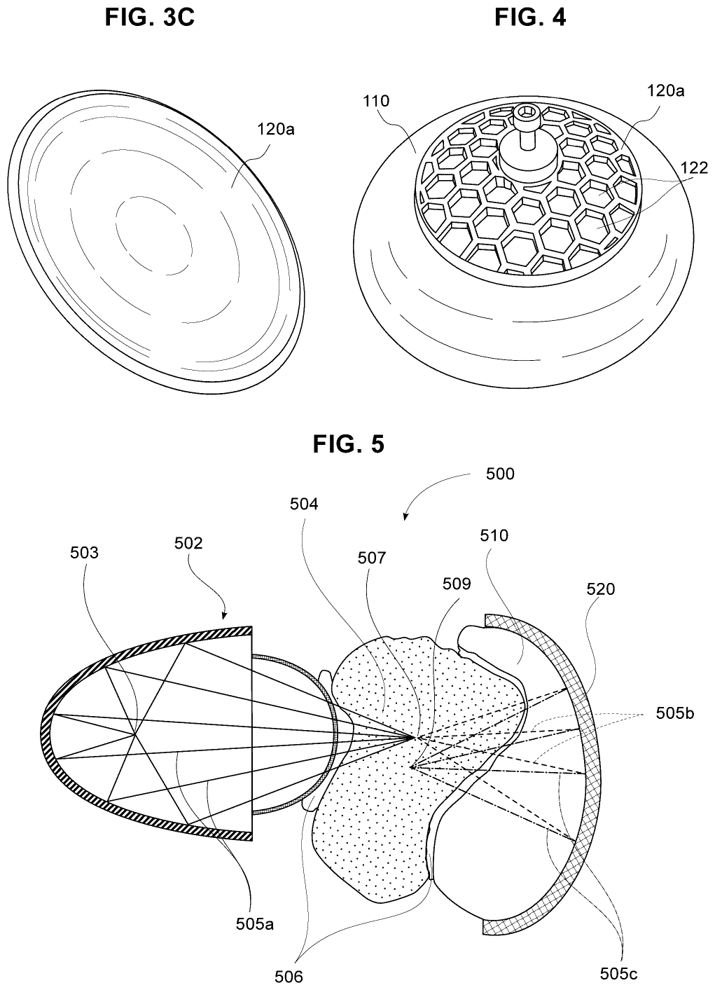

FIGS. 3A-3C show views of shockwave reflectors 120a, 120b according to an embodiment. A shockwave receiver 120 may be formed in different ways and/or made of different materials to facilitate different treatment goals. For example, as illustrated in FIG. 3A the shockwave receiver may be formed as a solid reflector 120a having an inner curvature (see FIG. 3C) selected to reflect and refocus the shockwave back at a predetermined point or range of points in the body part. In at least some types of treatment, the tensile/negative portion of the shockwave is undesirable. For example, when treating calcifications accessible with shockwave therapy (lithotripsy), the positive/compression portion of the shockwave compresses the calcification. The energy released breaks the calcium. On the other hand, the negative/tensile portion may cause cavitation gas bubbles, resulting in pain, and potentially hematoma. In electromagnetically and piezoelectrically generated shock waves, the negative/tensile wave is often as large as the positive part at lower energies. According to an embodiment, therefore, the reflector 120a may be formed of a material having a higher acoustic impedance than the impedance-controlled layer 110 to facilitate reflection of the shockwave in-phase with minimal (or no) change in the wave form. Materials having high acoustic impedance (significantly higher than 1.5 MRayl) include high-density materials such as certain metals (e.g., steel, tungsten, titanium, lead, and bismuth) and certain ceramics (e.g., barium titanate, zirconia, and alumina).

According to another embodiment, the shockwave reflector 120a may be formed of a material having a lower acoustic impedance to facilitate an inversion of the shockwave to enhance the tensile (negative) portion of the waveform. Materials having lower acoustic impedance than common soft-tissues or water (1.5 MRayl) include rubbers, some polymers, woods, and all gases. Using a shockwave reflector 120a of lower impedance compared to the impedance-controlled layer 110 facilitates an inversion of the wave pulse creating an enhanced tensile wave in a reflected secondary treatment pulse which can be further focused to surpass peak tensile wave pressures of the initial wave. This allows for secondary treatment with enhanced tensile forces. The geometry of a lower-impedance reflector may be substantially the same as a higher-impedance reflector 120a. Thus, a separate drawing is not provided. However, a higher-impedance reflector 120a and a lower-impedance reflector may be formed from materials that are visibly distinguishable or may be intentionally marked for distinction.

In some implementations, a desired amount of shockwave reflection may be obtained by using air as a reflector. Accordingly, as shown in FIGS. 3B and 4, an “air reflector” 120b having through-openings 122 throughout may be crafted to hold the impedance-controlled layer 110 while essentially providing minimal direct reflection.

In some embodiments, not shown, a shockwave receiver 120 may be formed using a mix of high-impedance and low-impedance materials in order to shape a reflection, provide a finer control of tensile wave properties, and/or direct reflections in a particular manner or to a particular location.

Those having skill in the art will recognize that it is also possible to avoid reflection altogether by providing sufficient impedance-controlled material to absorb and disperse the shockwave.

FIG. 5 is a cross-section diagram illustrating the shockwave trajectory intended in a shockwave therapy session using the disclosed system 500. A shockwave transducer 502 generates a general multi-directional wave from a source 503. The multidirectional wave is focused by reflecting from an interior curvature of the transducer 502 toward a subject body part 504 as a primary reflection 505a. The shockwave transducer 502 is acoustically coupled to the subject body part 504 via an acoustic gel (e.g., ultrasound gel) 506 that has an impedance substantially similar to the subject body part 504 and thus prevents loss of shockwave magnitude.

Some of the shockwave energy from the primary reflection 505a may be expended within the subject body part 504, chiefly at the treatment/focal zone 507 according to many implementations. A remainder wave 505b may traverse the subject body part 504 and enter an impedance-controlled layer 510 (such as the impedance matched layer 110 described above). The impedance-controlled layer 510 is coupled to the subject body part opposite the shockwave transducer 502. According to an embodiment, the impedance-controlled layer 510 may, like the transducer 502, be coupled to the subject body part 504 via acoustic gel (e.g., ultrasound gel, PVA, PEG, hydrogel or the like), while in other embodiments, the pouch or shell (e.g., 112) of the impedance-controller layer 510 may be sufficiently impedance-matched to maintain adequate acoustic coupling. The remainder wave 505b may traverse the impedance-controlled layer 510 and exit at a boundary thereof. According to an embodiment, a shockwave reflector 520 may receive the remainder wave 505b and reflect at least a portion thereof as a secondary reflection 505c. The secondary reflection 505c may be directed back to the treatment zone 507, at a secondary treatment location 509, or both, based on the type of reflector and treatment protocols.

Reflecting and refocusing of the shockwave allows for a rapid secondary transition of the secondary reflection 505c through the same treatment zone by aligning the wave direction, or increase the treatment volume by redirecting a secondary focal zone of the secondary reflection 505c into a secondary target zone 509. (Re) focusing allows for large peak pressure waves of a same order of magnitude as the initial treatment pulse. Here the geometry of the shockwave reflector 520 must be matched to primary geometry of the transducer 502, accounting for differences in distance from the intended treatment zone and desired secondary focal field geometry.

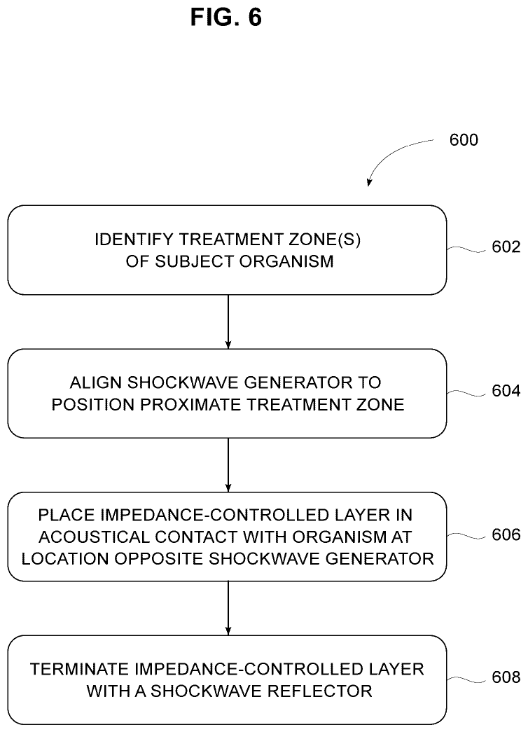

FIG. 6 illustrates a method 600 for using a secondary reflector in a shockwave therapy treatment system, according to an embodiment. A first operation 602 includes identifying at least one treatment zone of a subject organism. An operation 604 includes aligning an acoustic shockwave generator to a position proximate the identified treatment zone to direct and focus an acoustic shockwave to the treatment zone. Operation 606 includes placing an impedance-controlled layer in direct acoustical contact with the organism at a location opposite the acoustic shockwave generator across the treatment zone. An operation 608 includes terminating the impedance-controlled layer with a shockwave reflector having a predetermined fixed geometry. The predetermined fixed geometry may in some embodiments include a dimension greater than a wavelength of the acoustic shockwave. The shockwave reflector may be configured to reflect the acoustic shockwave and focus the shockwave on a secondary treatment zone.

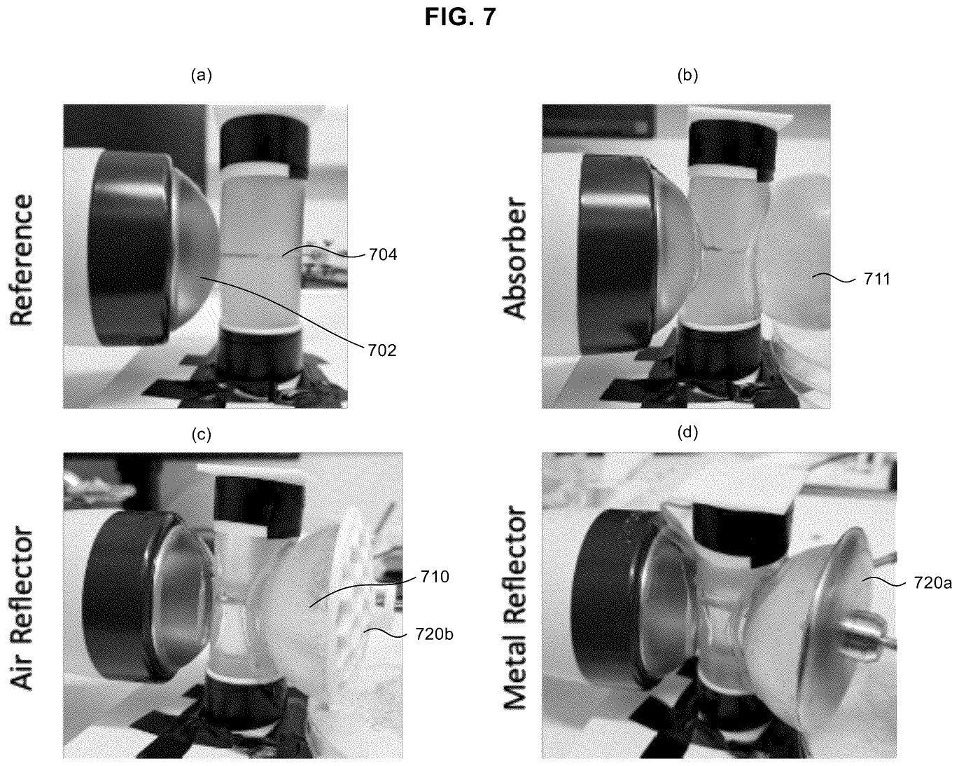

FIGS. 7-9 illustrate a testing setup and corresponding results in which a shockwave transducer 702 (such as transducer 102 described above) was applied to a simulated body part (704), according to an embodiment. In FIG. 7 a “Reference” configuration (a) includes only the shockwave transducer and simulated body part (e.g., comprising a gel or gelatin). An “Absorber” configuration (b) includes the features of the Reference configuration plus a shockwave absorber 711, e.g., a very thick impedance-controlled layer 710 with no reflector. An “Air Reflector” configuration (c) provides an impedance-controlled layer 710 and a shockwave terminator 720b (such as element 120b in FIG. 3B) configured to substantially provide an air/gas interface, permitting the shockwave to reflect from the air/gas interface. Finally, a “Metal Reflector” configuration (d) includes a metal reflector 720a with the impedance-controlled layer 710. In the (c) and (d) configurations of FIG. 7, it can be seen that the reflectors 720a, 720b are spaced from the “body part” 704 approximately the same as a distance from a shockwave generator internal to the transducer shown.

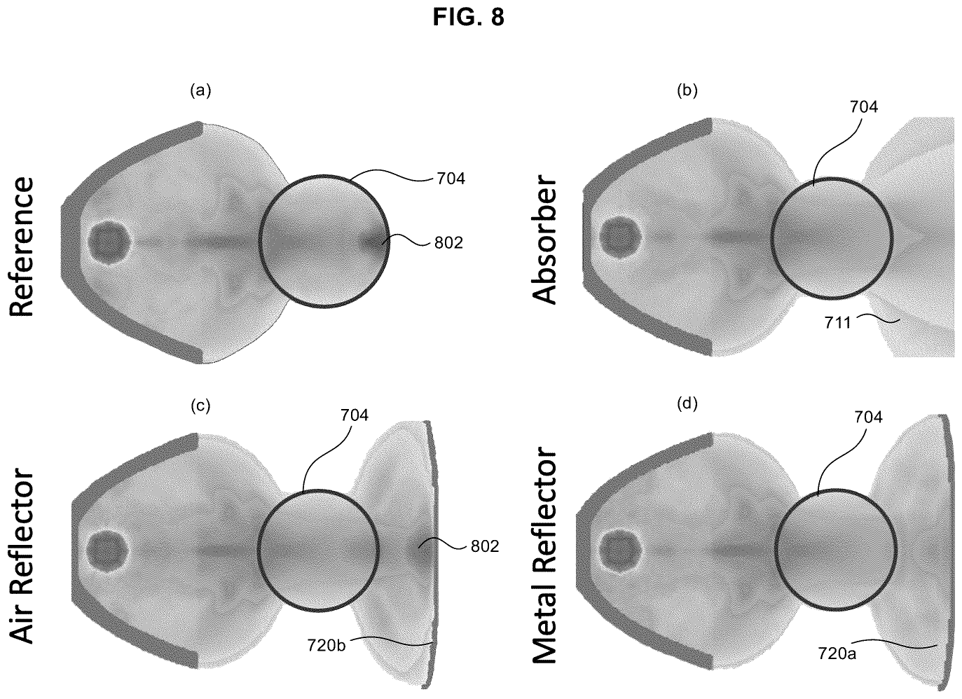

FIG. 8 depicts acoustic peak-pressure simulation results showing shockwave propagation corresponding to the different configurations from FIG. 7, according to an embodiment. In the reference configuration (a), a bright spot can be seen at the right side indicating a tensile/negative wave 802 within the simulated body part 704 (undesirable in certain procedures). In the absorber configuration (b) the absorber disperses the energy of the shockwave, eliminating the reflection which creates the enhanced tensile/negative portion of the wave. In the air absorber configuration (c), it can be seen that the negative/tensile portion 802 of the wave occurs at the air/gas interface at the right side, and is reflected back to a treatment zone within the simulated body part 704. Finally, in the metal reflector configuration (d), the reflector 720a returns an in-phase (i.e., non-inverted) shockwave energy to the treatment zone, permitting potential greater efficiency and effectiveness of the therapy.

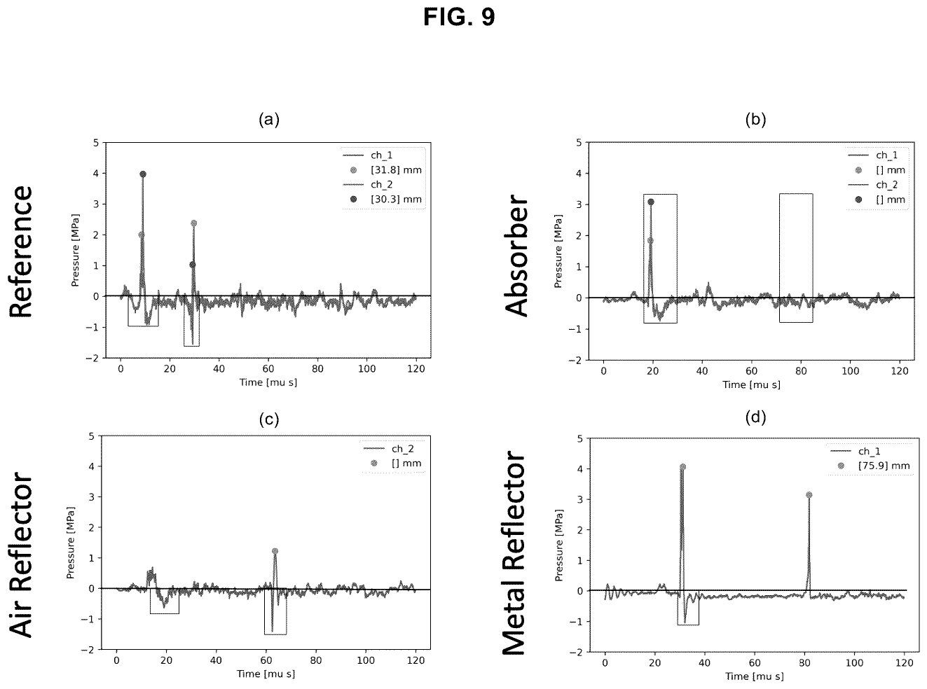

FIG. 9 includes respective graphs showing hydrophone-measured shockwave pressure vs. time at a treatment zone as measured in the different setups (a)-(d) of FIG. 7, according to an embodiment. In graph (a) of FIG. 9, the first pressure/tensile wave at about 8 μs is the primary/initial shockwave, and the pressure/tensile-inverted wave at approximately 30 μs is a reflection from the air/gas interface of the simulated body part 704. In configuration (b), the initial wave from is still seen, whereas no reflected shockwave is registered. The absorber 711 absorbed the shockwave and no reflection is seen. In configuration (c), the reflection of the shockwave wave is delayed to about 65 μs as the air/gas interface adds time, while the magnitude of the positive/pressure wave (and to a lesser degree the negative/tensile wave) is reduced in comparison to the reference configuration. However, the reflection of the air/gas interface has inverted the pressure profile resulting in a much larger negative/tensile wave on the shockwave's secondary pass. In configuration (d) the metal reflector 120a has reduced the negative/tensile wave by about 30% compared to the reference configuration, while increasing the magnitude of the reflected pressure wave by at least 70% compared to the reference, thus facilitating greater efficiency in the target zone or larger treatment volume.

It can be seen that a generated shockwave is targeted and acoustically coupled to treatment tissue.

The inventors recognize that the use of a reflector, as disclosed herein, may also permit treatment of tissues for which close access by a shockwave transducer is not practical. That is, a non-focused shockwave may be directed to a reflector 120 placed in an area inaccessible to the transducer. The reflector 120 can focus and direct the shockwave to the treatment area. For example, one or more reflectors may be placed within, e.g., a chest cavity, where it/they can focus and redirect shockwaves to an internal treatment target proximate the reflectors but distant from the transducer. According to another embodiment, a reflector could be temporarily or permanently implanted in the users to both reflect shockwaves for higher-efficiency EWST and/or to shield other body parts from the shockwaves.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Exemplary embodiments are shown and described in the present disclosure. It is to be understood that the embodiments are capable of use in various other combinations and environments and are capable of changes or modifications within the scope of the inventive concept as expressed herein. Such variations are not to be regarded as departure from the spirit and scope of the invention, and all such modifications as would be obvious to one skilled in the art are intended to be included within the scope of the following claims.

What is claimed is:

1. A shockwave therapy treatment system comprising:
a shockwave transducer configured to produce an acoustic shockwave and direct the shockwave to a treatment area of a patient body part;
an impedance-controlled layer configured to be conformable against the patient body part at a location across the patient body part from the shockwave transducer through the treatment area; and
a shockwave receiver positioned against the impedance-controlled layer and configured to receive at least a portion of the acoustic shockwave via the patient body part and the impedance-controlled layer and to affect one or more characteristics of the acoustic shockwave.

2. The shockwave therapy treatment system according to claim 1, wherein the impedance-controlled layer is non-gaseous.

3. The shockwave therapy treatment system according to claim 2, wherein the impedance-controlled, non-gaseous layer includes an elastomeric shell containing a cohesive liquid filler selected for high resistance to cavitation.

4. The shockwave therapy treatment system according to claim 3, wherein the cohesive liquid filler is a gel.

5. The shockwave therapy treatment system according to claim 1, wherein the shockwave receiver is selected from a set of shockwave reflectors each having a respective reflection profile directed to a different amount or direction of reflection of the acoustic shockwave.

6. The shockwave therapy treatment system according to claim 1, wherein the shockwave receiver is configured to reflect and refocus the shockwave in a predetermined direction and has an acoustic impedance higher than the impedance-controlled layer, the higher impedance of the shockwave receiver facilitating maintenance of compression and tensile wave elements of the reflected acoustic shockwave.

7. The shockwave therapy treatment system according to claim 1, wherein the shockwave receiver has a concave surface configured to reflect and refocus a portion of the shockwave in a predetermined manner and has an acoustic impedance lower than the impedance-controlled layer, the lower impedance facilitating an enhancement of a tensile wave element of the reflected acoustic shockwave

8. The shockwave therapy treatment system according to claim 1, wherein the shockwave receiver substantially exposes a boundary surface of the impedance-controlled layer to air, effectively resulting in an air reflector.

9. The shockwave therapy treatment system according to claim 1, wherein the shockwave receiver is formed of materials configured to absorb and disperse the received acoustic shockwave to de-energize and/or limit effective return of the acoustic shockwave to the patient body part.

10. The shockwave therapy treatment system according to claim 1, wherein the impedance-controlled layer is configured to substantially match the impedance of the body part.

11. The shockwave therapy treatment system according to claim 1, wherein the impedance-controlled layer is acoustically transparent.

12. The shockwave therapy treatment system according to claim 11, wherein the impedance-controlled layer is a gel.

13. An acoustic shockwave receiver device for use in shockwave therapies, the receiver device comprising:
an impedance-controlled layer, at least partly conformable against a user body part; and
a terminating reflector directly coupled to the impedance-controlled layer, the terminating reflector having a predetermined fixed geometry selected to receive and redirect to a treatment zone of the user body part at least a majority of an acoustic shockwave received through the user body part and the impedance-controlled layer.

14. The acoustic shockwave receiver device according to claim 13, wherein the impedance-controlled layer is non-gaseous.

15. The acoustic shockwave receiver device according to claim 14, wherein the impedance-controlled, non-gaseous layer includes an elastomeric shell containing a cohesive liquid filler selected for high resistance to cavitation.

16. The acoustic shockwave receiver device according to claim 15, wherein the cohesive liquid filler is a gel.

17. The acoustic shockwave receiver device according to claim 13, wherein the terminating reflector is formed of materials configured to absorb and disperse the received acoustic shockwave to de-energize and/or limit effective return of the acoustic shockwave to the user body part.

18. The acoustic shockwave receiver device according to claim 13, wherein the impedance-controlled layer is configured to substantially match the impedance of the user body part.

19. The acoustic shockwave receiver device according to claim 13, wherein the impedance-controlled layer is acoustically transparent.

20. The acoustic shockwave receiver device according to claim 19, wherein the impedance-controlled layer is a gel.

21. The acoustic shockwave receiver device according to claim 13, wherein the acoustic shockwave is received from a shockwave transducer applied to the user body part opposite the terminal reflector.

22. The acoustic shockwave receiver device according to claim 13, wherein the terminating reflector is a first shockwave reflector, a second shockwave reflector, or a third shockwave reflector, wherein:
the first shockwave reflector is configured to reflect the acoustic shockwave in a predetermined direction and has an impedance higher than the impedance-controlled layer, the higher impedance of the shockwave receiver facilitating in-phase maintenance of compression and tensile wave elements of the reflected acoustic shockwave;
the second shockwave reflector is configured to reflect a portion of the shockwave in a predetermined manner and has an impedance lower than the impedance-controlled layer, the lower impedance facilitating an enhancement of a tensile wave element of the reflected acoustic shockwave; and
the third shockwave receiver includes openings exposing the impedance-controlled layer to air, effectively providing an air reflector.

23. The acoustic shockwave receiver device according to claim 13, wherein the impedance-controlled layer has at least one compliant surface configured to deform for fitting closely against the user body part.

24. A method for treating a body part using acoustic shockwaves, the method comprising:
identifying at least one treatment zone of a subject organism;
aligning an acoustic shockwave generator to a position proximate the treatment zone to direct and focus an acoustic shockwave to the treatment zone;
placing a non-gaseous impedance-controlled layer in direct acoustical contact with the organism at a location opposite the acoustic shockwave generator across the treatment zone; and
terminating the impedance-controlled layer with a shockwave reflector having a predetermined fixed geometry, and configured to reflect the acoustic shockwave and focus the shockwave on a secondary treatment zone.

25. The method according to claim 24, wherein the secondary treatment zone is the same as the treatment zone.
